(19) United States
(12) Patent Application Publication
(10) Pub. No.: US 2026/0242460 A1
(43) Pub. Date: Aug. 20, 2026

(54) USE OF GOFLIKICEPT FOR THE TREATMENT OF IDIOPATHIC RECURRENT PERICARDITIS

(71) Applicant: R-Pharm Overseas, Inc., La Jolla, CA (US)

(72) Inventors: Mikhail Samsonov, Moscow (RU); Alexey Maslyansky, Saint Petersburg (RU); Sergey Grishin, Moscow (RU); Alexey Repik, Moscow (RU); Yan Lavrovsky, Del Mar, CA (US)

(21) Appl. No.: 19/296,878

(22) Filed: Aug. 11, 2025

Related U.S. Application Data
(63) Continuation-in-part of application No. 18/672,826, filed on May 23, 2024.
(60) Provisional application No. 63/345,142, filed on May 24, 2022.
(60) Provisional application No. 63/468,470, filed on May 23, 2023.

(51) Int. Cl. C07K 16/24; A61K 39/00; A61P 37/06

(52) U.S. Cl. CPC C07K 16/245; A61P 37/06; A61K 2039/505; A61K 2039/54; A61K 2039/545

(57) ABSTRACT

Disclosed invention provides for methods of treating idiopathic recurrent pericarditis (IRP), including a step of administering to a subject in need of treatment goflikicept at a therapeutically effective dose and an administration interval for a treatment period sufficient to reduce the risk of pericarditis recurrence relative to control. IL-1 inhibitor goflikicept shows efficacy and safety in patients with IRP. The goflikicept treatment allowed to discontinue other therapies for IRP, including glucocorticoids in the open-label treatment period, and reduced the risk of recurrence compared with placebo during the randomized withdrawal period.

### FIELD

Generally, the invention relates to the field of biological pharmaceuticals as well as their use in conditions associated with inflammatory disorders. More specifically, the invention relates to the use of goflikicept (RPH-104)—a heterodimeric IL-1R1/IL-1RAcP-derived composition for treating Idiopathic Recurrent Pericarditis.

### BACKGROUND

Idiopathic recurrent pericarditis (IRP) is a rare autoinflammatory disease (see, for example, Blank N, Lorenz H M. Current Rheumatology Reports (2018) 21:18; and https://www.orpha.net as assessed on Jan. 14, 2022) that has similar pathogenesis with adult-onset Still's disease and monogenic autoinflammatory diseases (see, for example, Imazio M. Heart. 2011 November, 97 (22) 1882-92; and Calabuig I J, Sanches Soriano R M, Marco Domingo T F et al. Rev Esp Cardiol. 2017, 70 (3): 208-219). IRP is diagnosed when standard diagnostic methods fail to identify a definitive etiology for pericardial disease and is characterized by cyclical episodes of symptom flare-ups followed by remission periods lasting 4 weeks or longer. IRP is a rare and understudied autoinflammatory disease, hindering the development of effective treatment strategies.

IL-1α and IL-1β are the pivotal cytokines in the pathophysiology of acute pericarditis and its recurrence. NLRP3 inflammasome overactivation has recently been implicated in the pathogenesis of IRP (Mauro, A. G., et al. The role of NLRP3 inflammasome in pericarditis: potential for therapeutic approaches. JACC Basic to Transl Sci 6, 137-150 (2021)). This overactivation results in the overproduction of interleukin (IL)-1 family proinflammatory cytokines, notably IL-1β, causing systemic symptoms, such as polyserositis, fever, and increased acute phase reactant levels, and IL-1a, suspected to promote chronic local inflammation within the pericardium, driving fibrosis and, ultimately, constriction (Weber, A., Wasiliew, P. & Kracht, M. Interleukin-1 (IL-1) pathway. Sci Signal 3, 1-7 (2010)). IL-1 blockade has demonstrated potential as a treatment for pericarditis (Abadie, B. Q. & Cremer, P. C. Interleukin-1 antagonists for the treatment of recurrent pericarditis. BioDrugs 36, 459-472 (2022)), including IRP. Currently, rilonacept (Arcalyst) is the only U.S. Food and Drug Administration-approved treatment for recurrent pericarditis that targets IL-1.

Goflikicept (RPH-104) (see US Patent Pub. No. US20210403534A1, published on Dec. 30, 2021, the entire teachings of which are incorporated herein by reference), a novel heterodimeric fusion protein designed to bind with high affinity to both human IL-1β and IL-1α, is a promising therapeutic approach (Myachikova, V. Y., et al. Treatment of idiopathic recurrent pericarditis with Goflikicept. J Am Coll Cardiol 82, 30-40 (2023)). The teachings herein describe an analysis of clinical trial with Goflikicept—a heterodimeric IL-1R1/IL-1RAcP-derived composition, which binds both, IL-1α and IL-1β, in patients with IRP.

In phase I safety studies, Goflikicept was well tolerated by healthy volunteers at doses up to 240 mg. Its favorable pharmacokinetic profile, with a half-life of 10 d, allows for convenient subcutaneous administration every 2 weeks. Therefore, its potential is being explored in IL-1-driven diseases such as IRP, gout, and familial Mediterranean fever, as well as in acute ST-segment elevation myocardial infarction (STEMI). In a phase II/III study, Goflikicept effectively prevented recurrences and maintained remission in patients with IRP, with an acceptable safety profile.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In certain aspects, the present invention provides for a method of treating recurrent pericarditis comprising administering subcutaneously to a patient in need of treatment, a loading dose of a Goflikicept at 240 mg, followed by a maintenance dose of Goflikicept at 80 mg once every two weeks.

In other aspects, the present invention provides for a method of treating recurrent pericarditis comprising administering subcutaneously to a patient in need of treatment, a loading dose of Goflikicept at 160 mg, followed by a maintenance dose of Goflikicept at 80 mg once every two weeks.

### BRIEF DESCRIPTION OF THE DRAWINGS

The following drawings and descriptions are provided to aid in the understanding of the invention:

FIG. 1 illustrates the study design: NSAIDs/colchicine/glucocorticoids were used as concomitant therapy in the run-in period; duration of Goflikicept monotherapy was 10 weeks in the run-in period (*among 22 enrolled patients non-loading dose regimen 80 mg every 2 weeks was applied in 10 patients, loading dose regimen 160 mg at week 0, 80 mg at week 1, 80 mg at week 2 followed by 80 every 2 week was applied in 12 patients in the run-in period; all responders were randomized into withdrawal period: GCs=glucocorticoids, PE=primary endpoint, NSAIDs=nonsteroidal anti-inflammatory drugs, WK=week, D=day);

FIG. 2 shows the flow diagram of the participants in the study: the initial dose regimen of Goflikicept was corrected after that event. Patients enrolled to the run-in period with recurrence (n=9) and in remission (n=13), at the end of the run-in period 20 patients randomized into double-blind withdrawal period in placebo and Goflikicept group in a ratio 1:1, 17 patients completed randomized withdrawal period and transferred to open-label extension study;

FIG. 3 illustrates chest pain occurrence, CRP and pericardial effusion during the run-in period: NRS scores for pain, CRP level, and pericardial effusion size were measured during the run-in period where all the patients received Goflikicept; chest pain, pericardial effusion size were assessed at baseline, on Day 3, at weeks 1,2,4 and thereafter every 4 weeks. CRP was measured at baseline, on Day 3, at weeks 1, 2 and thereafter every 2 weeks; vertical bars indicate the interquartile range (CRP=C-reactive protein; NRS=numerical rating scale; IQR=interquartile range; Wk=week);

FIG. 4 shows IL-6, IL-1RA and S100A8/A9 levels during the run-in period: biomarkers IL-6, IL-1RA and S100A8/A9 levels were measured at baseline, on Day 3, at Weeks 1, 2, 4 and thereafter every 4 weeks during the run-in period; vertical bars indicate the interquartile range (CLIA=chemiluminescence immunoassay, IL-6=Interleukin-6; IL-1RA=Interleukin-1 receptor antagonist, S100A8/A9=calprotectin, IQR=interquartile range, Wk=week);

FIG. 5 shows time to the pericarditis recurrence: Kaplan-Meier curves for the first pericarditis recurrence event after randomization in the randomized-withdrawal period are displayed; 9 (90%) patients in the placebo group had recurrence events, there were no recurrence events in the Goflikicept group; the median time to recurrence in the placebo was 49.5 (95% CI: 6 to 84) days and it could not be determined in the Goflikicept group (CI-Confidence Interval);

FIG. 6 illustrates chest pain occurrence, CRP and pericardial effusion during the run-in period for the dosing regiment with a 240 mg loading dose: NRS scores for pain, CRP level, and pericardial effusion size were measured during the run-in period where all the patients received Goflikicept;

FIG. 7 shows Single-cell transcriptome analysis of PBMCs in IRP:
a) Study design—Blood samples were collected from eight patients with IRP at three time points (days 0, 7, and 35),
b) Cell type annotation revealing the transcriptional landscape of integrated PBMCs from patients with IRP and public healthy control data;

FIG. 8 shows differential abundance analysis of PBMCs in patients with IRP and healthy donors:
a) Mean proportions of PBMC types during goflikicept treatment of IRP and in healthy donors,
b) Comparative analysis of PBMCs of healthy donors and patients with IRP before goflikicept treatment (upper panel), patients with IRP after 7 d of goflikicept treatment (middle panel), and patients with IRP after 35 d of goflikicept treatment (bottom panel) (ASDC, AXL+SIGLEC6+ dendritic cells; MAIT, mucosal-associated invariant T cells; NK, natural killer cells; DC, dendritic cells; DNT, double-negative T cells; moDC, monocyte-derived DCs; pDC, plasmacytoid DCs; tolDC, tolerogenic DCs; cDC, classical DCs);

FIG. 9 illustrates cell type response prioritization analysis in patients with IRP after goflikicept treatment, Only the cell types that passed the threshold of adjusted p-value<0.05 are shown:
a) Level 1 of cell type annotation,
b) Level 2 of cell type annotation,
c) Level 3 of cell type annotation;

FIG. 10 illustrates monocyte transcriptional response to goflikicept therapy:
a) Uniform manifold approximation and projection analysis of the expression of 33 genes related to IL1 inflammation, the results are presented sequentially in patients before goflikicept treatment, 7 days after treatment, 35 d after treatment, and in healthy donors,
b) Comparative analysis of IL1-related gene expression in monocytes during goflikicept therapy and in healthy donors. c. Intersection of DEGs detected in monocyte subsets on days 7 and 35 of treatment, the highest number of DEGs was found for classical monocytes on day 35.
d) DEGs in classical monocytes on day 35 of treatment vs. pre-treatment,
e) Changes in functional enrichment of classical monocytes on day 7 of treatment vs. pre-treatment,
f) Changes in functional enrichment of classical monocytes on day 35 of treatment vs. pre-treatment,
g) PROGENy pathway activity analysis of classical monocytes on days 7 and 35 vs. pre-treatment,
h) CollecTRI transcription factor activity analysis of classical monocytes on days 7 and 35 vs. pre-treatment. **P≤0.01, ***P≤0.001;

FIG. 11 shows B cell transcriptional response to goflikicept therapy:
a) Intersection of DEGs detected in B cell subsets on days 7 and 35 of goflikicept treatment,
b) DEGs in naïve B cells on day 35 of treatment vs. pre-treatment. c. DEGs in circulating plasma cell precursors on day 35 of treatment vs. pre-treatment,
d) Functional analysis of differential expression results for naïve B cells and circulating plasma cell precursors on day 35 vs. pre-treatment;

FIG. 12 illustrates T cell transcriptional response to goflikicept therapy:
a) Intersection of DEGs detected in T cell subsets on day 35 of goflikicept treatment,
b) DEGs in MAIT cells on day 35 of treatment vs. pre-treatment,
c) Functional analysis of differential expression results for MAIT cells on day 35 vs. pre-treatment,
d) DEGs in γδ T cells on day 35 of treatment vs. pre-treatment,
e) Functional analysis of differential expression results for γδ T cells on day 35 vs. pre-treatment; and

FIG. 13 shows positioning of goflikicept within the drug perturbation space defined by scRNA-seq and bulk RNA-seq data.

### DETAILED DESCRIPTION

The results of a clinical study of Goflikicept in patients with IRP are disclosed herewith. To identify potential new markers of disease activity a range of biomarkers was selected based on the pathogenesis of IRP, and their concentrations were analyzed before and after treatment with Goflikicept.

A double-blind, randomized-withdrawal, placebo-controlled study with open-label run-in period was designed. The study was conducted according to the principles of Declaration of Helsinki and the Good Clinical Practice guidelines and was approved by local ethics committees at each participating site. All patients signed informed consent.

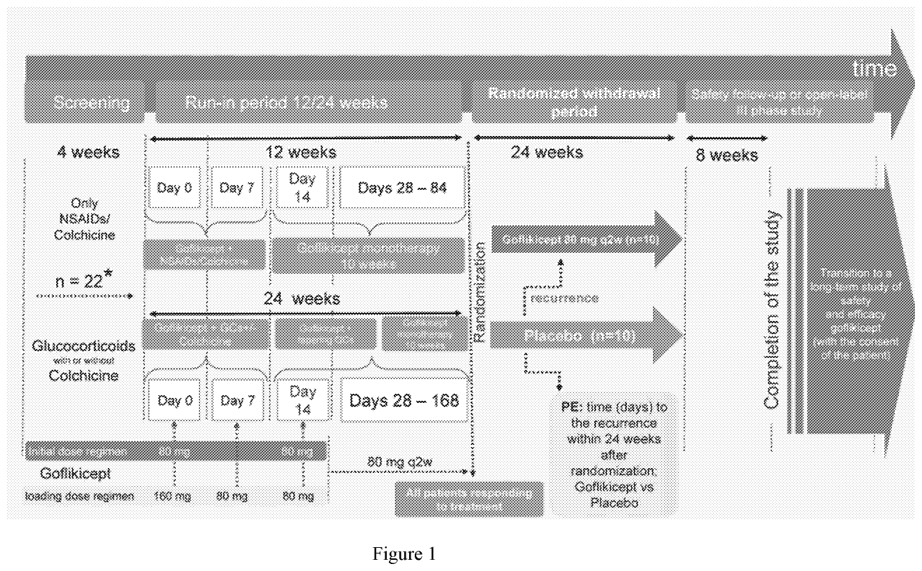

The study consisted of four periods: screening period up to 4 weeks; open-label run-in period of 12 weeks for treatment with NSAIDs and/or colchicine or 24 weeks for patients on treatment with glucocorticoids; randomized withdrawal (RW) period of 24 weeks; safety follow-up period of 8 weeks. In the end of RW period all patients who completed the study were offered to continue treatment in open-label extension study (FIG. 1).

For the criteria of response to therapy, the following signs were taken: a chest pain by numeric rating scale (NRS)≤3, CRP concentration≤5 mg/L, absent or mild (<10 mm) pericardial effusion on echocardiogram. The patients needed to achieve all three parameters to be assessed as responders on the fourteenth day of the therapy. Patients included to run-in period, had to continue standard of care treatment first 14 days at the same dosage they were taking at study entry. Responders in the NSAIDs/colchicine group, were stopped NSAIDs/colchicine on Day 14 without tapering, and were on goflikicept monotherapy for the rest 10 weeks of run-in period. Responders in the glucocorticoids/colchicine group, were stopped colchicine on Day 14 without tapering, then reduced and terminated glucocorticoids within next 12 weeks (on the combined treatment with goflikicept) and after that were on goflikicept monotherapy for the next 10 weeks.

At the end of the run-in, responders proceeded into the double-blind, placebo-controlled randomized-withdrawal period, when they received goflikicept 80 mg every 2 weeks or placebo in a ratio 1:1. In case of pericarditis recurrence during the randomized withdrawal period (assessed as the lack of response), the treatment group was unblinded.

Patients from the placebo group were retreated open-label therapy with goflikicept in the loading regimen (160 mg at Week 0, 80 mg at Week 1 and 80 mg at Week 2 thereafter 80 mg every 2 weeks within 60 weeks) (except one patient who received 80 mg SC without a loading dosage). Recurrence was defined as the development of at least two of the following signs: chest pain assessment score according to NRS>3 (and lack of any other possible reasons for increased pain intensity); CRP level>5 mg/L (and lack of any other potential reasons for CRP elevation); new or progression of existing pericardial effusion during the diastole as demonstrated by echocardiography.

Eligible patients, who completed the core study (run-in and RW periods) were offered to roll over to open label extension study for up to 24 months. Dose finding approach was applied during the run-in and randomized periods. Originally, the patients had administrated goflikicept 80 mg subcutaneously (SC) every 2 weeks. According to the clinical response, we decided to increase the initial dose and use next loading dose regimen: 160 mg at Week 0, 80 mg at Week 1, 80 mg at Week 2, followed by 80 mg every two weeks thereafter (look at section “DOSE FINDING”).

Adult (≥18 years) patients with IRP were eligible to participate in the study. Patients with recurrence or remission were enrolled in the run-in period. They had to take standard of care treatment (NSAIDs, colchicine and/or glucocorticoids) before enrollment and had to have at least one recurrence (which could be confirm at the office before signing informed consent). Patients with pericarditis that was of known etiology were excluded from the study on screening.

Echocardiography was performed according to the standard protocol proposed by the American Society for Echocardiography. Four standard views were used for reproducibility: subcostal, four-chamber, and parasternal long and short axis. 18 Patients assessed chest pain by using a numeric rating scale (NRS) with 11 points (from 0 to 10) where 0 and 10 corresponds to the absence of or unbearable pain. The quality of life was assessed using the questionnaire SF-36 (Medical Outcome Short Form (36) Health Survey—a short form of health assessment that includes 36 points). The questionnaire was completed by the patients at scheduled visits before any other procedures. Biomarkers included IL-1α, IL-1β, interleukin-1 receptor antagonist (IL-1RA), IL-6, IL-18, tumor necrosis factor alpha, S100A8/A9 (calprotectin) and S100A12 (calregulin).

Primary endpoint was the time to the first pericarditis recurrence in the RW period. The secondary endpoints included change from baseline in chest pain scores, CRP and biomarkers levels, pericardial effusion size, proportion of patients with response, proportion of patients with glucocorticoid withdrawal at 12 weeks; change of quality-of-life parameters.

Safety assessment consisted of collection and assessment of adverse events, physical examination, and laboratory tests. For patients who discontinued goflikicept therapy during the run-in period, randomized withdrawal period, at the end of randomized period and did not transfer to open label extension study, safety data was collected during 8 weeks after the last dose of goflikicept.

Sample size was estimated to be 20 patients that would be sufficient to obtain a study power of 90%, considering the type I error at the level of 0.05, and assuming placebo effect at 20% and goflikicept at 80%. For statistical analysis, all values are reported as median and interquartile range (IQR). All analysis were performed on an intention-to-treat basis without imputation for missing data. Event-free survival was estimated by the Kaplan-Meier method. A log-rank test was used to compare the time to recurrence within 24 weeks after randomization. The Fisher exact test was used to assess rate of recurrence in the randomized withdrawal period. The differences between treatment groups were computed using the Mann-Whitney U test for continuous variables. Significance of the change from baseline was tested using paired Wilcoxon signed-rank test. All tests were 2-tailed, and a p-value<0.050 was considered statistically significant. All statistical analyses were performed using SAS version 9.4 (SAS Institute).

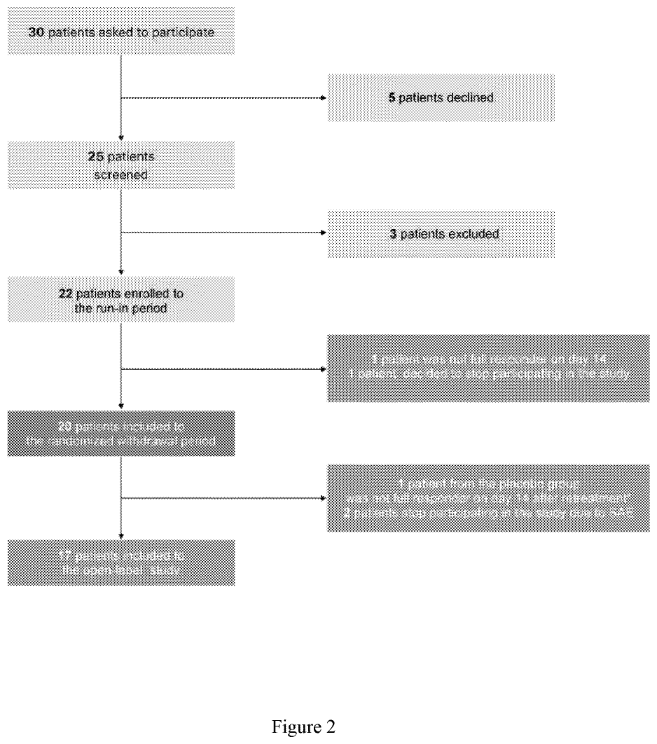

Twenty-five patients were screened, and 22 patients were enrolled in the study. Table 1 shows the demographic and baseline characteristics. Nine patients had recurrence, and 13 patients were in remission at the time of enrollment. 20 patients completed the run-in period and proceed to the RW. Two patients did not complete the run-in period: one patient (4.5%) due to lack of response at Day 14, one patient (4.5%) decided to stop participating in the study (FIG. 2).

Two dosing regimens were used in this study. The initial dose regimen (80 mg every 2 weeks) was re-evaluated based on goflikicept treatment response in two patients (one patient in run-in and one patient in RW periods, FIG. 2). The following loading dose regimen of goflikicept was applied: 160 mg at Week 0, 80 mg at Week 1, 80 mg at Week 2, followed by 80 mg every two weeks thereafter. Same regimen was used in case of pericarditis recurrence in patients receiving placebo during RW period. There were no failures in recurrence resolution on loading dose regimen compared to the initial dose regimen of goflikicept. Importantly, after randomization all patients who had no recurrence received goflikicept in the same dose regimen of 80 mg every 2 weeks.

Comparison of two dose regimens is challenging due to heterogeneity of patient groups received goflikicept in these two regimens. In the group of patients receiving 80 mg every 2 weeks there were 6/10 people with recurrence at enrollment. In the group of patients with the loading dose regimen, only 3/12 of the study participants had a recurrence at the enrollment. A two-fold difference in the proportion of patients with recurrence at the study enrollment determined by different baseline levels of following parameters: median, (IQR): on Pain Scale-4.0 (3.0-7.0) and 2.5 (0.0-3.5) points; CRP 7.95 mg/L (1.90-11.00) and 1.70 mg/L (1.0-5.60); pericardial effusion 0.0 mm (0.0-7.0) and 3.0 mm (0.0-14.00) for the non-loading and loading dose regimen groups, respectively.

TABLE 1
Demographic and other baseline characteristics

| Run-in period | Randomized withdrawal period | |
|---|---|---|
| Total | Goflikicept | Placebo |
| n = 22 | n = 10 | n = 10 |

Sex, n (%)

| | | | | | | |
|---|---|---|---|---|---|---|
| Male | 5 | (22.7) | 2 | (20.0) | 2 | (20.0) |
| Female | 17 | (77.3) | 8 | (80.0) | 8 | (80.0) |
| Age (years), median (IQR) | 48.5 | (37.0-58.0) | 47.5 | (33.0-56.0) | 47.5 | (40.0-50.0) |
| Disease duration (years), | 0.6 | (0.1-2.0) | 0.6 | (0.2-1.7) | 0.6 | (0.1-2.2) |
| median (IQR) | | | | | | |
| Pericardial effusion (mm), | 1.0 | (0.0-7.0) | 0.0 | (0.0-3.0) | 0.0 | (0.0-0.0) |
| median (IQR) | | | | | | |
| CRP (mg/L), median (IQR) | 2.95 | (1.30-9.70) | 0.65 | (0.50-0.90) | 1.20 | (0.90-3.50) |
| Chest pain, median (IQR) | 3.0 | (1.0-4.0) | 0.0 | (0.0-1.0) | 0.0 | (0.0-1.0) |

Previous therapy, n (%)

| | | |
|---|---|---|
| NSAIDS and/or colchicine | 20 | (90.9) |
| Glucocorticoids | 2 | (9.1) |

The number of pericarditis recurrences (n, %)

| | | | | | | |
|---|---|---|---|---|---|---|
| 1 recurrence | 4 | (18%) | 1 | (10%) | 2 | (20%) |
| 2 recurrences | 8 | (36%) | 3 | (30%) | 5 | (50%) |
| ≥3 recurrences | 10 | (46%) | 6 | (60%) | 3 | (30%) |

CRP = C-reactive protein;
IQR Interquartile range;
NSAIDs = nonsteroidal anti-inflammatory drugs

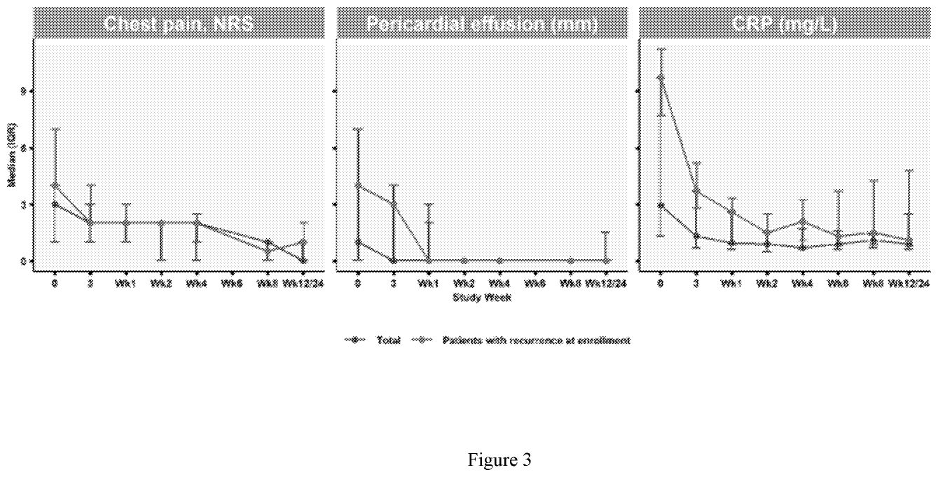

Treatment response was achieved in 21 of 22 (95.5%) enrolled patients, 8 of 9 (88.9%) patients enrolled with recurrence. All patients enrolled without recurrence remained in remission at Day 14. There were no new recurrence events on goflikicept therapy, despite NSAIDs/colchicine/glucocorticoid discontinuation during the run-in period. Two patients on glucocorticoid therapy discontinued within 12 weeks and were on goflikicept monotherapy by the end of run-in period. There was quick reduction of CRP levels accompanied by reduction of chest pain and pericardial effusion compared to baseline during run-in treatment period (12/24 weeks) in the enrolled patients (n=22) (FIG. 3). More pronounced reduction of CRP, NRS, pericardial effusion compared to baseline was demonstrated in patients with recurrence at enrollment (n=9). The highest median chest pain levels (4.0 (4.0-7.0) in NRS) at baseline were seen in this group patients. There was statistically significant decrease of pain levels by Day 3 compared to baseline (p=0.03), and the level continued to decrease progressively reaching (1.0 (0.0-2.0) (p=0.009) by the end of run-in period (Week 12/24).

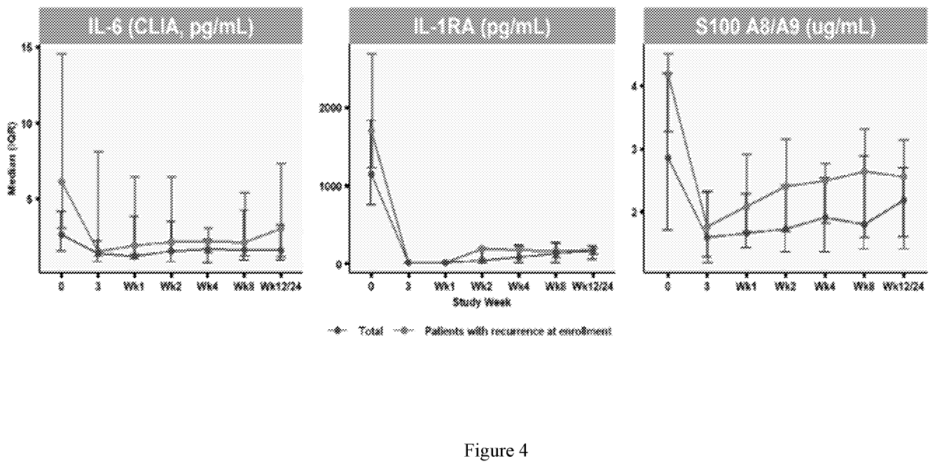

The highest median CRP levels at baseline were found in the group of patients with recurrence at enrollment (9.70 (7.70-11.20) mg/L). Median CRP levels reduced on Day 3 (3.70 (2.80-5.20) mg/L, p=0.001). Statistically significant decrease in median CRP levels was shown during 8 weeks, and at the end of run-in period (Week 12/24) (1.10 (0.80-4.80) mg/L, p=0.028) (FIG. 3). The size of the pericardial effusion consistently decreased during the run-in period with statistically significant difference comparing to the baseline by Day 3 (p=0.027) and Day 14 (p=0.002) and positive changes maintained from Week 4 and up to the end of the run-in period (Week 12/24; p=0.020). The median (IQR) value of size of the pericardial effusion was 4.0 mm (0.0-7.0) and 0.0 mm (0.0-0.0) in patients with recurrence at baseline and Week 12/24 respectively. Decreases of IL-1RA, interleukin-6 (IL-6), S100A8/A9 levels were reported during the run-in period, thus confirming anti-inflammatory effect of goflikicept. A decrease in IL-1RA levels compared to baseline was demonstrated for the run-in period (in the overall patient population). All patients demonstrated statistically significant decrease in IL-1RA level by the end of the run-in period (Week 12/24 (p<0.001). IL-1RA level change was similar in patients enrolled in the study with the baseline recurrence of the disease and those with no recurrence at enrollment (p=0.05 for both cohorts compared to baseline). A decrease in IL-6 levels compared to baseline was demonstrated during the run-in period. The decrease in IL-6 levels was statistically significant by 4 weeks of the run-in period (p<0.05) compared to baseline. In patients with baseline recurrence, IL-6 levels significantly decreased from Day 3 and up to Week 4 (p<0.05). S100A8/A9 levels decreased comparing to baseline during the run-in period, 2.86 (1.64-4.19) μg/ml, 2.18 (1.59-2.78) μg/ml for baseline and Week 12/24 respectively. In patients with baseline recurrence, calprotectin levels started to decrease from the first week of the run-in period (p<0.05) and continued to decrease by the end of run-in period. The patients with baseline recurrence demonstrated statistically significant decrease in S100A8/A9 level by the end of run-in period (Week 12/24) (p=0.001) (FIG. 4). Other biomarkers did not demonstrate clear differences compared to baseline.

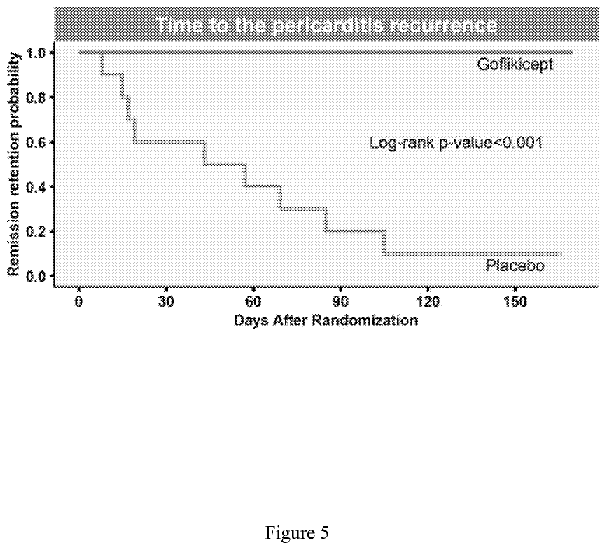

A total of 20 patients were randomized. Recurrence of pericarditis occurred in 9 of 10 patients in placebo group, there were no recurrence events in goflikicept group within 24 weeks after randomization (p<0.001) (Central Illustration). The median time (95% CI) to pericarditis recurrence in patients randomized to placebo group was 49.5 (6 to 84) days (FIG. 5). After retreatment with goflikicept and recurrence resolution, there were no new recurrence events. The distribution of recurrence by combined criteria is shown in the Table 2.

Regarding the secondary endpoints, statistically significant difference of changes in Physical functioning scale score and IL-1RA levels were shown in goflikicept group compared to placebo group. Median (IQR) Physical functioning scale score was 75.00 (60.00-85.00, n=10 patients) and 79.99 (75.00-90.00, n=10 patients) in goflikicept group at baseline and on Day 56 respectively. The median (IQR) Physical functioning scale score was 80.00 (65.00-95.00, n=10 patients) and 77.50 (57.50-90.00, n=4 patients) in placebo group at baseline and on Day 56 respectively. The data indicate statistically significant improvement of this SF-36 domain in goflikicept group compared to placebo group (p<0.02). The median change from baseline on Day 56 was 4.99 (−5.00-9.99) and −15.00 (−17.51-(−7.50) in goflikicept and placebo group respectively. The median (IQR) IL-1RA levels were 169.97 (113.43-213.37, n=10 patients) pg/mL and 152.37 (15.00-283.78, n=10 patients) pg/mL in goflikicept group at baseline and on Day 56 respectively. The median (IQR) IL-1RA levels were 145.14 (15.00-429.14, n=10 patients) pg/mL and 875.60 (423.41-1231.46, n=4 patients) in placebo group at baseline and on Day 56 respectively. The data indicate statistically significant difference of this parameter in goflikicept group compared to placebo group (p<0.009). The median change from baseline on Day 56 was −9.13 (−55.40-59.39) and 765.53 (408.41-967.78) in goflikicept and placebo group respectively. The other parameters in the goflikicept group did not demonstrate statistically significant differences compared to the placebo group.

TABLE 2
Distribution of recurrence definition by combined criteria

Number of
patients with
recurrence atRW
period N = 9

Individual criterion

| | |
|---|---|
| Chest pain NRS score >3 | 9 (100.0%) |
| New/progression of existing pericardial effusion | 6 (66.7%) |
| CRP >5 | 4 (44.4%) |

Combined criteria (based on recurrence definition)

| | |
|---|---|
| Chest pain NRS score >3 + New/progression of | 5 (55.6%) |
| existing pericardial effusion | |
| Chest pain NRS score >3 + CRP >5 | 3 (33.3%) |
| Chest pain NRS score >3 + CRP >5 + New/ | 1 (11.1%) |
| progression of existing pericardial effusion | |

Abbreviations:
CRP = C-reactive protein; value mg/L,
NRS = numericalrating scale;
RW—randomized withdrawal
CRP = C-reactive protein; value mg/L,
NRS = numerical rating scale;
RW—randomized withdrawal

Overall, during the study 122 adverse events were reported in 21 patients (95.5%). Adverse events occurred in 15 of 22 patients (68%) during the run-in period; In RW period, adverse events occurred in 5 of 10 patients (50%), 9 of 10 (90%), 8 of 9 patients (89%) in placebo, goflikicept and goflikicept retreatment group respectively. Table 3 shows the adverse events of special interest occurred throughout the study. Five serious adverse events were reported in 4 patients. All the serious adverse events occurred during the RW period: these included coronavirus disease (COVID-19) associated pneumonia in one patient from the goflikicept group; pericarditis recurrence in one patient from the placebo group; COVID-19 associated pneumonia and sepsis (in one patient each), severe bilateral pneumonia in one patient after goflikicept retreatment in the placebo group. There were no deaths during the study. Three of 22 patients (13.6%) were identified as anti-drug antibody positive and one of these was identified as neutralizing antibody positive. Among these patients the treatment failed due to different causes, one patient withdrew informed consent in run-in period, and two patients were randomized to placebo in RW period and experienced recurrence. These patients were enrolled in the study with recurrence. These patients received goflikicept in the non-loading dose regimen of 80 mg every 2 weeks. No anti-drug antibodies were detected in 12 patients who received the loading dose regimen.

The management of IRP can be challenging due to lack of sensitive biomarkers for assessing the activity, and limited therapeutic options. On these premises, we conducted a clinical trial with a new IL-1 inhibitor—goflikicept—in patients with IRP. IL-1 inhibitors currently available on the market include monoclonal anti-IL1-β antibody canakinumab, recombinant human IL-1RA anakinra and fusion protein rilonacept12. Goflikicept is a fusion protein with the structure different from rilonacept. Treatment with goflikicept significantly reduced the pericarditis recurrence rate compared with both the placebo and the allowed withdrawal of standard of care therapy (including glucocorticoids in all patients). Patients with IRP achieved remission within the first 14 days after the start of therapy. Two patients on glucocorticoids were able to completely discontinue treatment without the risk of recurrence. The therapy was well tolerated; the adverse events were predictable based on the mechanism of action of the drug and were not different from class-specific side effects of other IL-1 inhibitors. Local reactions at the injection site were recorded in 18.2% of patients. The study started at the beginning of the pandemic of the new coronavirus infection (COVID-19), one patient on goflikicept was withdrawn from the study due to that serious adverse event. Antidrug antibodies were detected in three (13.6%) patients in the 80 mg dosing regimen. However, no antibodies were detected in patients receiving goflikicept using the loading dose regimen. Being a homodimer, rilonacept carries two identical functional units in each polypeptide chain of the molecule: sequentially cloned extracellular domain of IL1 receptor 1 (IL-1-R1) and extracellular domain of IL1 receptor accessory protein (IL-1-RAcP). Therefore, each molecule of rilonacept has two domains of IL-1-R1 and two domains of IL-1-RAcP. Goflikicept is a heterodimeric protein in which one IL-1-R1 domain is placed on one polypeptide chain of the molecule while one IL-1-RAcP domain is placed on another polypeptide chain. Thus, goflikicept has a significantly smaller molecular mass (~150 kDa vs. rilonacept's~250 kDa) leading to a simpler and less expensive manufacturing process. Goflikicept binds to IL-1β with the constant of dissociation (KD) of 11.8 fM as shown by

surface plasmon resonance approach, while reported KD of rilonacept and IL-1β was in 1-2 pM range. 19 Half-life of goflikicept is longer than one of rilonacept allowing less frequent (once every two weeks) administration. As it was previously shown in PHAPSODY study, IL-1 blockade is an effective approach in treatment of recurrent pericarditis. In this study, rilonacept significantly reduced risk of recurrence. Same as in RHAPSODY, in the present study run-in and randomized withdrawal periods to access the efficacy were used. There were several differences between the present study and RHAPSODY. In in the present study, patients with or without recurrence were enrolled, whereas only patients with recurrence were enrolled in the RHAPSODY. In the present study, NSAIDs/colchicine were stopped immediately without tapering in responders on Day 14, whereas in RHAPSODY NSAIDs/colchicine dose had to remain unchanged until Week 4 and then tapered within 6 weeks of the run-in period. In our study, goflikicept monotherapy lasted for 10 weeks, while rilonacept monotherapy was 2 weeks in the run-in period. In the present study the predefined criteria of pericarditis recurrence were used: investigator and Principal investigator assessed clinical response. In RHAPSODY, recurrence was adjudicated by an Independent Review Committee based on predefined criteria.

The results of both studies indicated high efficacy of the IL-1 inhibitors in treatment of recurrent pericarditis. There were no recurrence events in the goflikicept study and only 2 events in the rilonacept study due to temporary interruption of the trial-drug regimen. Biomarkers of the disease activity. In the present study the spectrum of candidate inflammatory biomarkers were included as secondary endpoints, well discovered in other autoinflammatory diseases. The following potential biomarkers were identified-IL-6, IL-1RA, S100A8/A9 that relevant to pathogenesis of IRP, and significantly reduced during the treatment. The concentration of TNF-alpha did not change, which emphasizes the fact that IRP is an “IL-1-driven” disease. IL-1α was not detected in serum, which proves that this cytokine is local and does not appear in the systemic circulation. 22 IL-1β was not increased in the studied groups. Considering that the cytokine works within ultra-low concentrations, is rapidly destroyed and is better detected in plasma than in serum, we assume that the obtained result does not refute the connection of the cytokine with the pathogenesis of the disease, but confirms the methodological difficulties of its analysis. Moreover, the reflection of IL-1β production is an increase in IL-1RA concentration, which in our study was evaluated in all patients both in patients with high and low CRP concentrations. Its simplicity of detection, long half-life, and work within high concentrations makes this biomarker one of the potential indicators of disease activity. S100A8/A9 and S100A12 usually reflect subclinical inflammation in other autoinflammatory diseases. In the present study, the concentration of S100A12 did not significantly change, while S100A8/A9 already decreased to normal on the third day after treatment. It should be noted that, as in the case of IL-1RA, its values were higher than normal in patients with low CRP concentration. The limitation of using these biomarkers (as well as CRP) is their low specificity for infectious events they increase in response to any trigger activating the innate immune system. This may partly explain the tendency for S100A8/A9 to increase over time, as the study was conducted during the pandemic. Thus, this suggests the need not to consider them separately from the clinical picture, but to use them as additional indicators to confirm or refute the presence of disease activity. Current data suggest that interleukin-1 inhibitors could be an option after failure of NSAIDs/colchicine combination and before glucocorticoids in order to avoid their side effects. The findings with goflikicept confirm the central role of IL-1, and expand the armamentarium of drugs that can effectively treat IRP.

To further evaluate the efficacy and safety of Goflikicept treatment in patients with IRP, subjects with recurrence (n=9) or in inter-recurrence period (n=13) were enrolled in the run-in period of 12 weeks for treatment with NSAIDs and/or colchicine or 24 weeks for treatment with corticosteroids (CS). Dose finding approach was applied, where subjects from the first cohort (n=10) received subcutaneous Goflikicept, 80 mg every 2 weeks, subjects from the second cohort (n=12)—240 mg load within the first week, 80 mg within the second week, thereafter 80 mg every 2 weeks on top of background NSAIDs, colchicine and/or CS. Treatment response was considered as an achievement or maintenance of the following criteria: chest pain NRS≤3, CRP≤5 mg/L, absent or mild (<10 mm) pericardial effusion on Day 14. In responders NSAIDs/colchicine were stopped immediately, CS therapy was reduced and terminated within 12 weeks and for the next 12 weeks patients were on Goflikicept monotherapy. At the end of the run-in responders proceeded into the double-blind, placebo-controlled randomized-withdrawal (RW) period, and received 80 mg of Goflikicept every 2 weeks, or placebo. Primary endpoint was the time to the first pericarditis recurrence. In case of recurrence, the patients were unblinded, patients from placebo group retreated with Goflikicept. According to the analysis plan, efficacy/safety analysis included only unblinded data during the RW period.

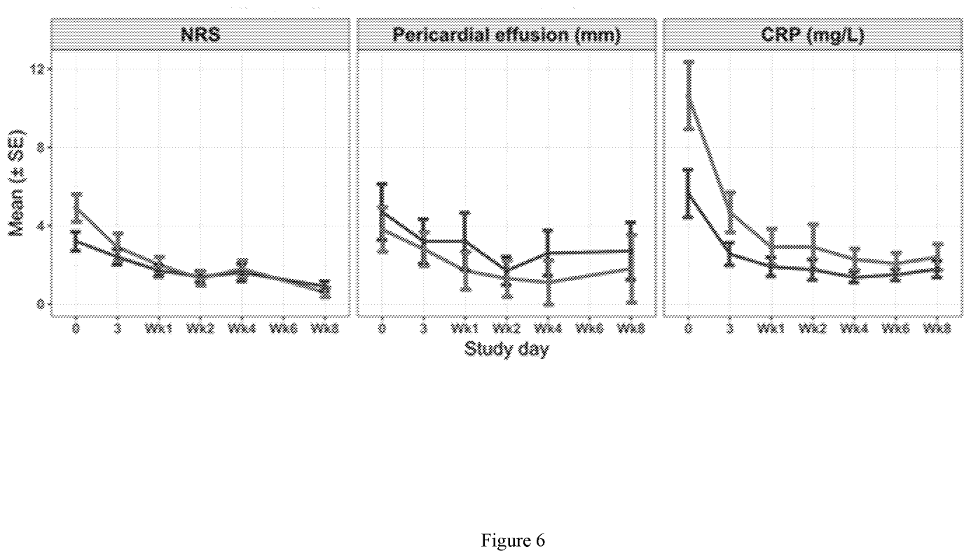

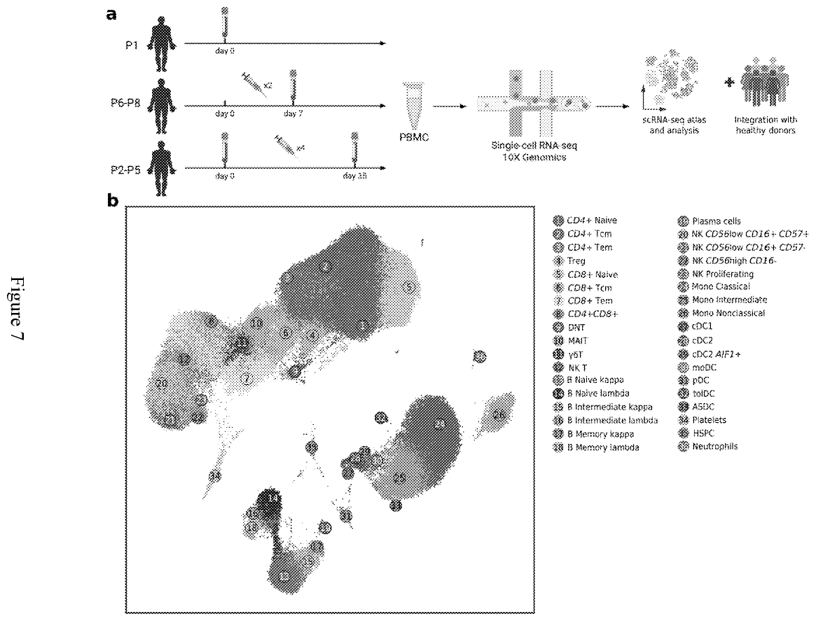

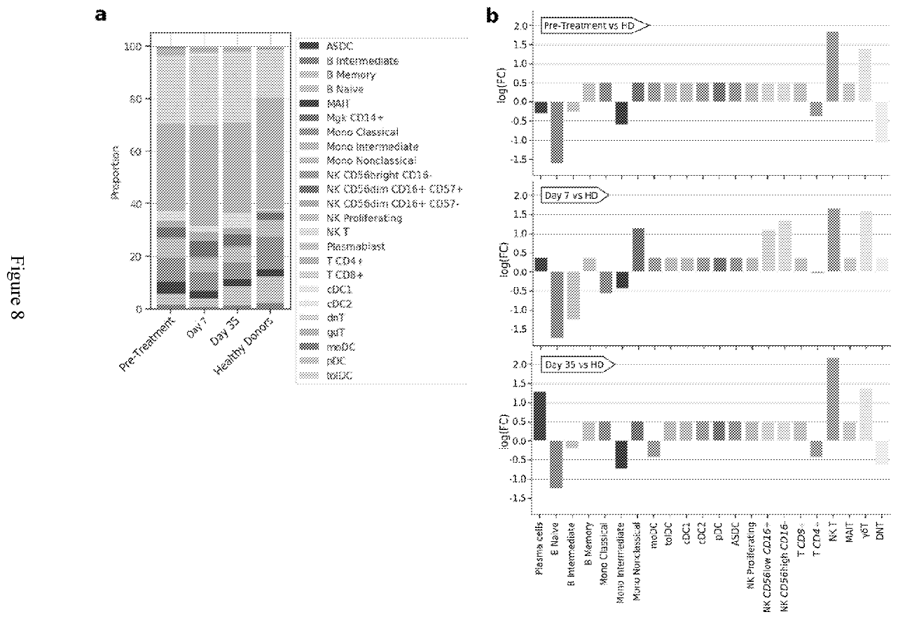

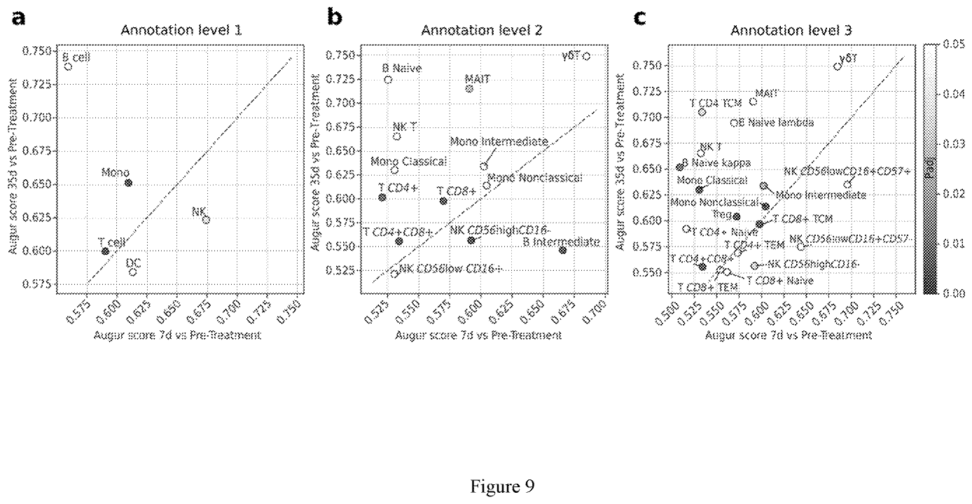

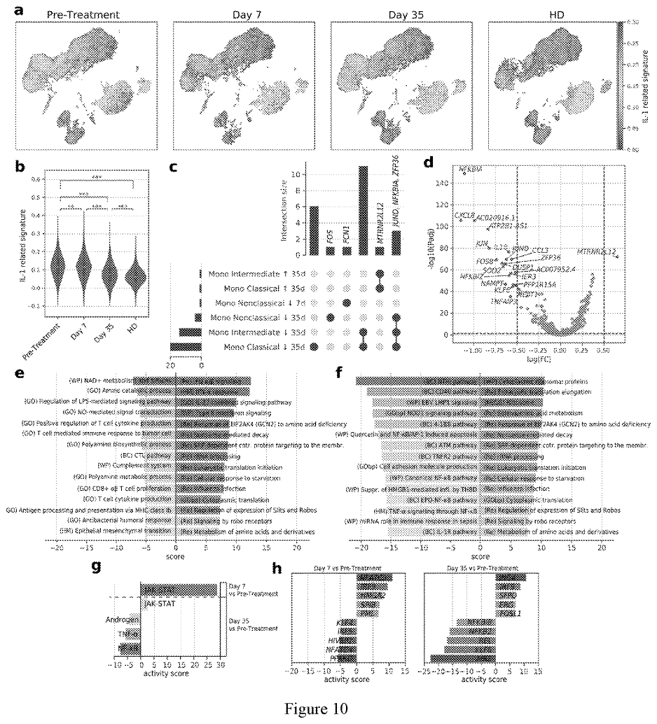

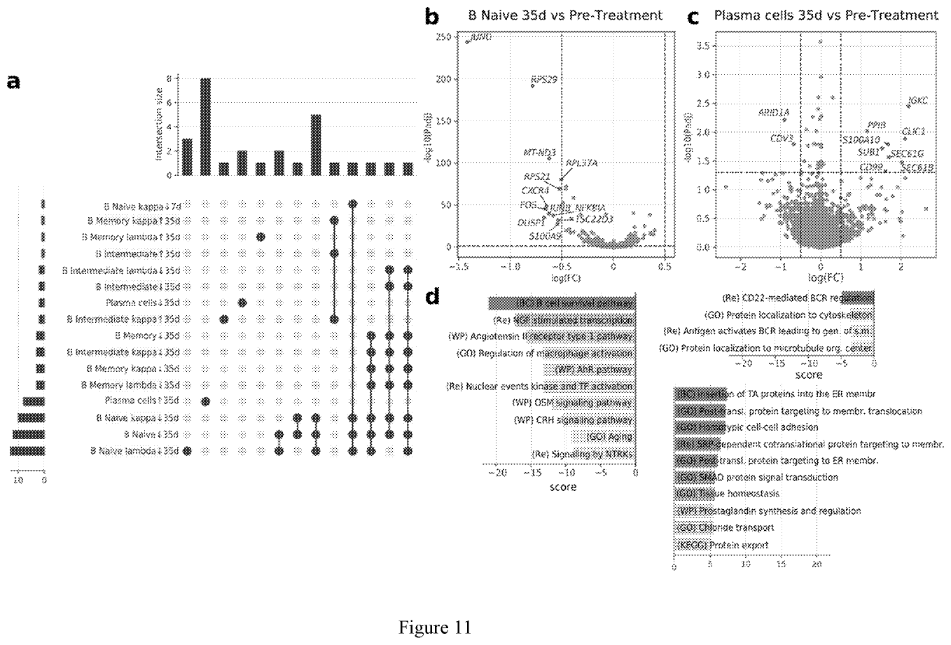

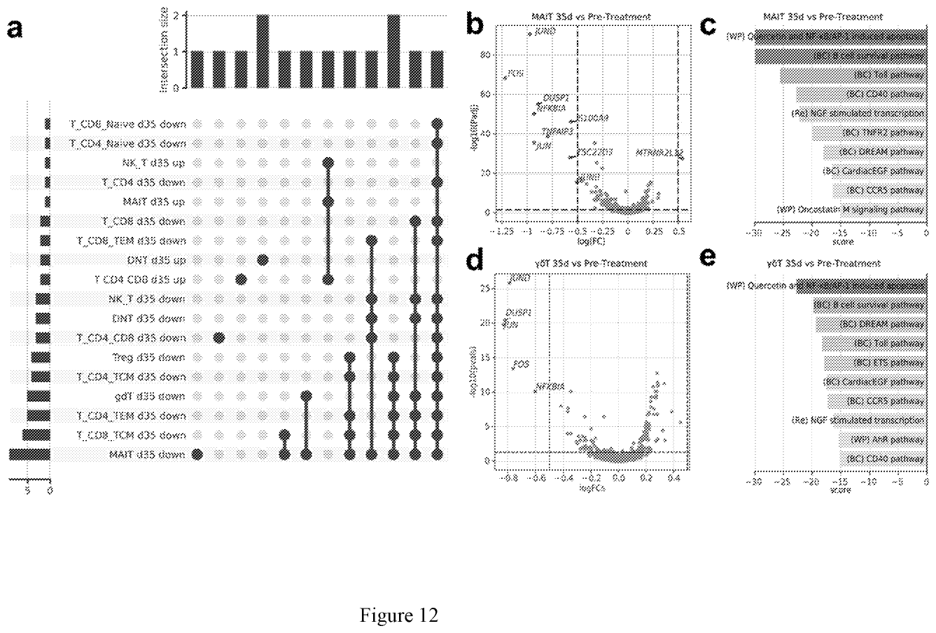

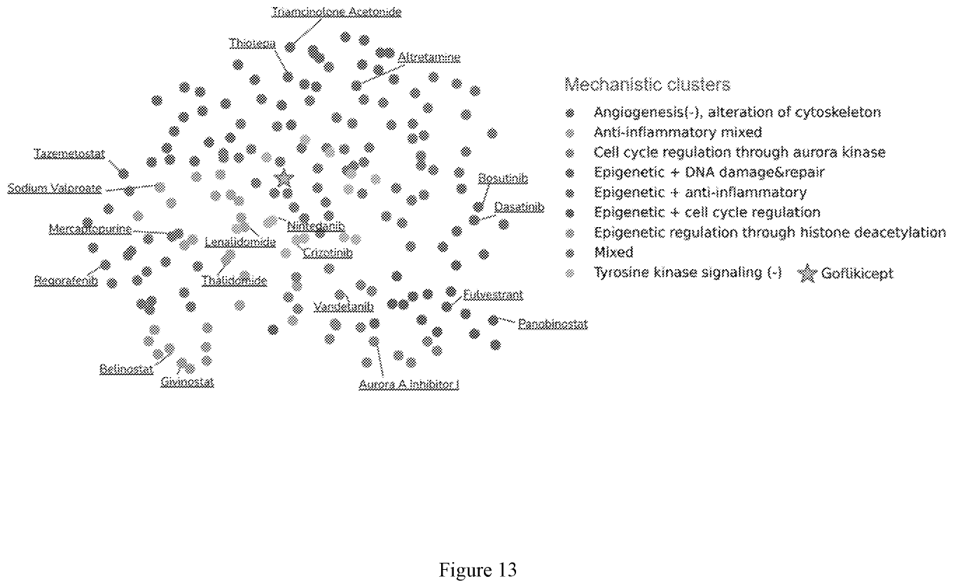

Treatment response was achieved in 8 out of 9 patients enrolled with recurrence, and all patients
enrolled without recurrence remained in remission at Day 4. There were no new recurrences
events on Goflikicept therapy, despite NSAIDs/colchicine/CS discontinuation during the run-in period. In the run-in period mean NRS, CRP and pericardial effusion decreased from baseline (FIG. 6). Decrease of concentrations of IL-1RA, IL-6, calprotectin was reported during the run-in period, which confirms anti-inflammatory effect of Goflikicept. A total of 20 patients were randomized. Recurrent pericarditis occurred in 8 out of 10 patients in placebo group, there were no recurrence events in Goflikicept group up to the time of the analysis. After retreatment with Goflikicept and recurrence resolution there were no new recurrence events. Adverse events (AEs) occurred in 15 of 22 patients (68.2%) during the run-in period. During the run-in period, 6 out of 22 patients (27%) experienced infections, 4 (18.2%) cholesterol elevation, 3 (13.6%) lymphopenia, 2 (9.1%) lipase elevation, 2 (9.1%) injection site reactions. AEs occurred in 4 out of 8 patients (50%) during placebo treatment in the RW period. AEs occurred in 6 out of 8 patients (75%) after retreatment with Goflikicept. There were no deaths and no new safety signals. Overall safety profile was similar to those described for other IL-1 blockers.

The data show ability to achieve and maintain IRP remission on Goflikicept monotherapy with a favorable risk-benefit ratio.

### Example 1: Effect of IL-1 Blockade on Peripheral Blood Mononuclear Cell Populations in Idiopathic Recurrent Pericarditis

While excessive IL-1 production is a hallmark of IRP, the specific molecular pathways responsible for this dysregulation remain largely unknown. Further, a detailed investigation of the mechanism of action of Goflikicept would provide an understanding of how IL-1 inhibition affects the immune system. Specifically, examining the effects of Goflikicept on innate and adaptive immune cells may reveal novel therapeutic targets for autoimmune and autoinflammatory conditions characterized by IL-1 overactivity. The effects of Goflikicept on peripheral blood mononuclear cell (PBMC) were investigated in a subset of transcriptomes in patients with IRP using single-cell transcriptome analysis. The goal of the investigation was to identify potential molecular mechanisms underlying treatment response and provide novel insights into the fine-tuned regulation of cellular pathway activity in both target and off-target PBMC subsets during disease and treatment.

PBMCs isolated from blood samples of eight patients with IRP were subjected to single-cell RNA sequencing (scRNA-seq) using the 10× platform (FIG. 7A). For one patient, a sample was collected only at baseline (day 0). For three patients, samples were collected on day 7 after receipt of two doses of Goflikicept. For the remaining four patients, samples were collected on day 0 and day 35 after receipt of four doses of Goflikicept. After quality control, excluding cells of insufficient quality and likely doublets, 86,743 cells were retained for analysis, including 36,012 cells from day 0, 20,761 cells from day 7 of Goflikicept treatment, and 29,970 cells from day 35.

To comparatively investigate PBMC heterogeneity in patients with IRP and healthy donors, Harmony was used to integrate our IRP single-cell transcriptome data with publicly available single-cell data from healthy donor PBMCs on the 10× Genomics website (https://support.10×genomics.com/single-cell-gene-expression/software/downloads/latest). This integrated dataset, comprising 142,375 cells, was subjected to uniform manifold approximation and projection dimensionality reduction, Leiden clustering, and manual and automatic annotation, resulting in the identification of eight major cell types at the first level of annotation: T cells, B cells, monocytes, natural killer (NK) cells, dendritic cells (DCs), neutrophils, platelets, and hematopoietic stem/progenitor cells (FIG. 7B). Clusters of neutrophils, platelets, and hematopoietic stem/progenitor cells were removed from subsequent analysis because they were not targeted during sample preparation.

Monocytes were subclustered into classical, intermediate, and nonclassical monocytes (FIG. 7B). In the T cell cluster, CD4+ T (T helper [Th] cells), CD8+ T (T cytotoxic cells), CD4+ CD8+ T (double-positive T [DPT] cells), and CD4low CD8low T (double-negative T [DNT] cells) were identified. CD4+ T cells comprised four subtypes: T-regulatory, naïve, central memory, and effector memory, while CD8+ T cells comprised three subtypes: naïve, central memory, and effector memory. Additionally, mucosal-associated invariant T (MAIT) cells, gamma-delta (γδ) T cells, and NKT cells were identified. These T cell subtypes are consistent with findings in recent studies on peripheral T cells. NK cells were subclustered into CD56high CD16−, CD56low CD16+, and proliferating NK cells. CD56low CD16+NK cells were subdivided into CD57+ and CD57-subsets. B cells were subclustered into naïve B cells, intermediate B cells, memory B cells, and plasma cells. Naïve, intermediate, and memory B cells were further subdivided into subsets with increased expression of IGKC or IGLC2. DCs were subclustered into type 1 and type 2 conventional, plasmacytoid, AXL+ SIGLEC6+, monocyte-derived and, presumably, highly-activated tolerogenic DCs.

We next investigated differences in cell type abundance between patient samples from all time points and healthy donors (FIG. 8). No clusters exclusive to IRP samples were identified. The consistent presence of all cell clusters across all time points suggested robust data integration and reproducibility. Compared to healthy donor samples, pre-treatment samples from patients with IRP showed increased abundances of CD8+ T cytotoxic cells and MAIT cells and a decreased abundance of CD4+ T helper cells (FIG. 8A). Comparative subset analysis revealed a significant increase in the abundances of NKT cells and γδ T cells, along with a decrease in those of naïve B and DNT cells in pre-treatment samples from patients with IRP compared to healthy donor samples (FIG. 8B, upper row). After 7 d of Goflikicept treatment, these differences remained largely consistent, except for DNT cells, which were increased at this time point. The populations of nonclassical monocytes, NK CD56bright CD16− cells, and NK CD56dim CD16+ cells were expanded, whereas those of intermediate B cells were decreased (FIG. 8B, middle row). After 35 d of Goflikicept treatment, the abundance of plasmablasts was increased. The relative abundances of naïve and intermediate B cells, NKT cells, and γδ T cells remained relatively stable compared to those in healthy donor samples (FIG. 8B, bottom row).

We used Augur to assess cell type prioritization in the transcriptional response to Goflikicept treatment on days 7 and 35. Transcriptional changes were more pronounced on day 35 than on day 7, with an increased number of cell types exhibiting higher Augur scores (FIG. 9). At the first level of annotation, only NK cells showed substantial perturbation on day 7, whereas monocytes, DCs, T cells, and, particularly, B cells, were perturbed on day 35 (FIG. 9A). The second level of annotation revealed that perturbations on day 35 were the most prominent in γδ T cells, MAIT cells, and all major monocyte subsets (FIG. 9B). Detailed analysis revealed perturbations on day 35 in naïve CD4+ T helper cells, naïve B cells, and intermediate and classical monocyte subsets (FIG. 9C). Non-classical monocyte subsets, NK cell subsets, and NKT cells were significantly perturbed after 7 d of Goflikicept treatment.

Given the known mechanism of action of Goflikicept, we initially focused on the IL-1β response. We compiled a signature of 33 genes associated with IL-1 and inflammation, which was altered particularly evidently in monocytes (FIG. 10A). The IL-1 signature score was the highest in monocytes from untreated patients with IRP and decreased with treatment to nearly the levels in healthy donor monocytes (FIG. 10B). The core set of downregulated differentially expressed genes (DEGs; |log 2FC|≥0.5, adjusted P≤0.05) on day 35 of treatment was shared between classical and intermediate monocytes (FIG. 10C). On day 35 of treatment, most genes in classical monocytes were downregulated, with only one gene being upregulated (FIG. 10D). On day 7 of treatment, the interferon (IFN) response was significantly upregulated in classical monocytes, whereas downregulated responses were weakly expressed and exhibited a heterogeneous pattern. By day 35, the IFN response was reverted, and pathways associated with the inflammatory response, tumor necrosis factor alpha (TNF-α) signaling via nuclear factor kappa beta (NF-κB), and the IFN-γ response were strongly downregulated. Consistent herewith, PROGENy analysis showed that on day 7, only the Janus kinase/signal transducers and activators of transcription (JAK/STAT) pathway was upregulated. However, by day 35, JAK/STAT pathway upregulation was reduced, concurrent with the downregulation of the NF-κB, TNF-α, and androgen pathways (FIG. 10G). CollecTRI analysis revealed that transcription factor activity was downregulated significantly more strongly on day 35 than on day 7. This substantial downregulation involved key inflammatory and IL-1-related genes, including AIRE, NFKBIB, and RELB (FIG. 10H). Functional enrichment analysis corroborated these findings, revealing a significantly more pronounced effect of Goflikicept treatment on day 35 than on day 7. The decrease in activity was associated with a wide range of physiological and pathological processes, predominantly inflammatory in nature, as well as with processes mediated by IL-1 and TNF (FIGS. 10E and F).

Among all B cell subsets, naïve B cells and circulating plasma cell precursors had the highest number of DEGs on day 35 compared to pre-treatment (FIG. 11A). The majority of DEGs in naïve B cells were downregulated on day 35 (FIG. 11B), whereas plasma cell precursor DEGs were predominantly upregulated (FIG. 11C). Hallmark gene set enrichment analysis revealed that several pro-inflammatory pathways, including TNF-α signaling via NF-κB, were downregulated in naïve B cells on day 7 of treatment, whereas the IFN-α response was upregulated. Concurrently, TGF-β, hypoxia, and TNF-α were downregulated, whereas the JAK/STAT was upregulated as indicated by PROGENy pathway activity analysis. CollecTRI analysis of transcription factor activity on day 7 revealed numerous up- and downregulated genes in naïve B cells. In contrast, on day 35, only FOS was upregulated, whereas JUN, RUNX1, DDIT3, JUNB, JUND, and STAT2 were markedly downregulated. Conversely, plasma cell precursors demonstrated increased activity, with upregulation of MEF2C, SOX2, HOXA7, PBX1, ZEB1, CXXC1, and RBPJ. Functional enrichment analysis corroborated these findings, revealing more pronounced effects of Goflikicept treatment on day 35 in both naïve B cells and plasma cell precursors than in other cell types (FIG. 11D). While in naïve B cells, numerous pathways were significantly downregulated, in plasma cell precursors, most pathways were upregulated.

On day 35 following Goflikicept treatment, changes in T cell subset responses were observed. Specifically, genes in T helper, T cytotoxic, and T regulatory cell subsets were strongly downregulated. Gene upregulation was less frequent and primarily observed in NKT, MAIT, CD4+ CD8+ T, and DNT cells. The most pronounced downregulation, affecting a relatively large number of genes, was detected in T regulatory cells, central memory and effector memory T helper cells, central memory T cytotoxic cells, MAIT cells, and γδ T cells (FIG. 12A).

As γδ T and MAIT cells demonstrated the most pronounced changes among T cell subtypes on day 35 (FIG. 9 and FIG. 12), we proceeded to investigate this relatively rare T cell subset in greater detail. Response patterns were similar between γδ T and MAIT cells but more pronounced in the latter. DEGs were found only on day 35 compared to pre-treatment, which corresponds with Augur prioritization. On day 35, MAIT cells exhibited significant downregulation of eight genes (JUND, FOS, DUSP1, NFKBIA, JUN, S100A9, TSC22D3, and JUNB) (FIG. 12B), whereas in γδ T cells, five genes were downregulated (JUN, DUSP1, JUND, FOS, and NFKBIA) (FIG. 12D). The only pathway downregulated in γδ T cells on day 7 was the TNF-α pathway. Hallmark gene set enrichment analysis revealed the downregulation of numerous pathways related to cell survival, cytokine production, cell interaction, and migration, including NF-κB/AP-1-induced apoptosis, B cell survival, Toll signaling, CD40 signaling, and CCR5 signaling in γδ T and MAIT cells on day 35 of therapy (FIG. 12C and FIG. 12E).

To determine the relationships between the effect of Goflikicept and those of known drugs, we used perturbation modeling (FIG. 13). First, we trained a model on a diverse set of scRNA-seq (sci-Plex3, three cell lines treated with 188 small-molecule drugs) and bulk RNA-seq (LINCS1000) datasets. The trained model captured the effects of various drugs, as reflected in their gene expression profiles, within a latent space of drug perturbations. Next, we clustered this latent space to identify groups of drugs exhibiting similar effects. Strikingly, Goflikicept clustered alongside tyrosine kinase inhibitors, non-steroidal anti-inflammatory drugs, glucocorticosteroids, and anticancer drugs, indicating potential similarities in their mechanisms of action or effects.

### Methods

### Patient Characteristics

In total, eight patients with IRP were enrolled in the study. The diagnosis of IRP was based on 2015 European Society of Cardiology guidelines on the management of pericardial diseases. Given that IRP is a diagnosis of exclusion, we ruled out chronic infections (including tuberculosis), systemic rheumatic diseases, encompassing both autoimmune (e.g., connective tissue diseases) and autoinflammatory (e.g., adult-onset Still's disease, Schnitzler syndrome) disorders, and monogenic autoinflammatory diseases (e.g., familial Mediterranean fever, TNFRSF1A-associated periodic syndrome, MVK deficiency, and NLRP3-associated diseases). The median age of the eight patients was 49.5 years (interquartile range, 43.75-55.25, standard deviation=14.1). To assess disease activity, we used a modified Pouchot score and the Systemic Disease Inflammatory Index to evaluate systemic symptoms associated with IRP. The main characteristics of the patients and samples included in the study are presented in Table 1.

The eight patients with IRP showed a poor response to colchicine treatment and subsequently received biologic therapy. All patients were classified into two groups based on the degree of inflammation: low (N=2 patients) or high (N=6 patients). Seven patients (P2-P8) received goflikicept during the clinical trial NCT04692766, and one patient (P1) received corticosteroids on the day of sample collection.

Two samples were collected (during relapse and remission) from patients P2-P5, one sample during relapse from patient P1, and one sample during remission from patients P6-P8. The following criteria were used to define remission: (1) chest pain score≤3 on the numeric rating scale, (2) C-reactive protein concentration≤5 mg/L, and (3) absent or mild pericardial effusion (<10 mm) on echocardiography. Patients were considered responders on day 7 or day 35 of therapy if all three criteria were met. Patients were not selected based on any presumed likelihood of treatment response. Nevertheless, all patients who received goflikicept in this study responded to therapy.

This study was conducted in accordance with the Declaration of Helsinki and was approved by the local Ethics Committee of the Almazov National Medical Research Center (protocol number 28, dated 12 Feb. 2018). Written informed consent was obtained from all study participants.

TABLE 1
Characteristics of the patients and samples analyzed in the present study.

| | | | | | | | | Modified | C- | | | |
|---|---|---|---|---|---|---|---|---|---|---|---|---|
| | | | | | Days | Total | | Pouchot | reactive | | | |
| Patient | Sample | | Age | Weight | of | dosage, | Dose, | activity | protein, | Ferritin, | IL-6, | IL1RA, |
| ID | ID | Sex | years | kg | therapy | mg | mg/kg | score | mg/L | ng/mL | ng/mL | ng/mL |
| P1 | SC8 | M | 70 | 85 | 0 | | | 4 | 1.80 | ND | ND | ND |
| P2 | SC5 | M | 37 | 89 | 0 | 0 | 0 | 4 | 10.6 | 93.23 | 1.89 | 832 |
| P2 | SC9 | M | 37 | 89 | 35 | 320 | 3.6 | 0 | 1.3 | 87.72 | 2.34 | 30 |
| P3 | SC3 | M | 74 | 83 | 0 | 0 | 0 | 2 | 3.5 | 96.72 | 4.21 | 966 |
| P3 | SC11 | M | 74 | 83 | 35 | 320 | 3.9 | 1 | 0.7 | 58.28 | 3.69 | 30 |
| P4 | SC10 | F | 56 | 80 | 0 | 0 | 0 | 1 | 1.6 | 75.51 | 2.84 | 554 |
| P4 | SC14 | F | 56 | 80 | 35 | 400 | 5.0 | 0 | 0.4 | 47.15 | 0.81 | 30 |
| P5 | SC12 | F | 55 | 75 | 0 | 0 | 0 | 3 | 2 | 81.23 | 3.97 | 2319 |
| P5 | SC13 | F | 55 | 75 | 35 | 320 | 4.3 | 1 | 1.6 | 52.97 | 2.74 | 306 |
| P6 | SC4 | F | 50 | 109 | 7 | 160 | 1.5 | 0 | 0.7 | 13.89 | 3.82 | 30 |
| P7 | SC7 | M | 49 | 90 | 7 | 160 | 1.8 | 0 | 2.3 | 289.60 | 4.4 | 30 |
| P8 | SC2 | F | 26 | 84 | 7 | 160 | 1.9 | 0 | 0.9 | 26.85 | 1.21 | 30 |

ND—data not available

### PBMC Isolation

Blood samples were collected from one patient before treatment (day 0), from three patients on day 7 (post two-dose goflikicept), and from four patients on both days 0 and 35 (post four-dose goflikicept), for a total of 12 samples (Table 1). Peripheral vein blood samples were collected in vacuum tubes with K3EDTA (Vacutest, KIMA, Arzergrande, Italy). PBMCs were isolated using SepMate™-15 tubes (StemCell Technologies, Vancouver, CA) and Ficoll-Paque PLUS density gradient medium (Cytiva, Marlborough, MA, USA) according to the manufacturer's recommendations. In brief, 4 mL of density gradient medium was carefully transferred into a SepMate™-15 tube. The diluted blood sample (3 mL of blood and 3 mL of PBS) was pipetted down the side of the tube. Tubes were centrifuged at 1,200×g, room temperature for 10 min. The top layer containing the enriched PBMCs was collected, and the cells were washed with PBS and centrifuged at 300×g for 8 min.

scRNA-seq

Single-cell capturing and library construction were performed using the Chromium Controller and Single Cell 3′ Reagent Kit v.3.1 (10× Genomics) according to the manufacturer's protocol and as previously described. In brief, approximately 100,000 PBMCs per sample were loaded onto 10× Genomics Single Fluidics chips to generate gel beads in emulsion, which contained oligonucleotide-barcoded beads for single-cell indexing and unique molecular identifiers (UMIs) for transcript quantification. Uniquely barcoded RNAs were reverse transcribed followed by cDNA amplification in a Veriti™ 96-Well Thermal Cycler (Applied Biosystems). Libraries were barcoded with Chromium™ i7 Multiplex Kit dual indexes, pooled in equimolar ratios, and sequenced on an Illumina NextSeq 2000 system.

Single-cell capturing and library construction were performed using the Chromium Single Cell 3′ v3 according to the manufacturer's instructions (10× Genomics). Raw sequencing reads were aligned to GRCh38 (GENCODE v32/Ensembl 98), and feature-barcode matrices were generated using Cell Ranger v.6.1.2.

scRNA-Seq Data Analysis

### Data Processing

To process the scRNA-seq data, we mostly followed the standard scRNA-seq analysis pipeline implemented in Scanpy (v.1.10.1), a Python package for single-cell analysis, using Python (v.3.12). We also used scRNA-seq data of PBMCs from 16 healthy donors from the 10× genomics official website (https://www.10×genomics.com/). First, we removed genes expressed in fewer than two cells and filtered out low-quality cells based on the number of detected genes, mitochondrial UMI fraction, and total UMI count. We retained cells with gene counts between 400 and 3,500 and mitochondrial UMIs<15%. Subsequently, we used Scrublet in Scanpy to identify doublets among the PBMCs from the patients and healthy donors. We used the default parameters for scanpy.pp.scrublet (i.e., sim_doublet_ratio=2, synthetic_doublet_umi_subsampling=1, n_prin_comps=30), set random_state to 42, and threshold to 0.25. We thus detected 3,079 doublets in the patients with IRP, and 604 in the healthy donors. Following filtering, the data were normalized to the median total count and log 1p-transformed. The new IRP dataset and public healthy donor dataset were integrated using HarmonyPy (v.0.0.10) over dataset, patient, and condition batches.

### Cell Type Annotation

Using the integrated data, we performed unsupervised clustering with the Leiden clustering algorithm. Highly expressed genes within each subset were identified using the Wilcoxon rank test implemented in Scanpy. Automatic cell type annotation was performed using Azimuth and CellTypist and compared with manual annotation. To annotate cell types on deeper levels, we ran subclustering for each of the cell type from level 1 (the broadest levels).

We subclustered monocytes, T cells, B cells, DCs, and NK cells at a resolution of 0.5, −3, −1.5, −0.3, and −0.5, respectively. The expression markers used to differentiate the cell types in the final annotation are listed in Supplementary Table 1. During annotation, we identified two small subclusters of classical monocytes that expressed the markers of classical monocytes along with platelet or T cell markers. These two clusters had a relatively high mean doublet score (>0.1) and were deemed to be clusters of doublets or aggregated cells and therefore excluded from analysis. Similarly, a subcluster of B cells that exhibited mixed expression of naïve B cell and T cells markers and had a relatively high mean doublet score (>0.2) was removed.

### Bioinformatic and Statistical Analyses

Differential abundance analysis was performed using scCODA algorithm available in the package pertpy (v.0.10.0). Response prioritization across cell types was conducted using Augur.

Differential gene expression analysis of goflikicept-treated conditions compared to the untreated was performed using MAST-RE. We refrained from comparing expression profiles between the healthy control and IRP conditions (untreated or treated) due to the inability to exclude batch effects from different datasets while preserving authentic biological information. We did not identify any significant sex, age or weight-related differences across untreated or treated IRP patients' samples. GSEA was based on a univariate linear model using decoupler and P-values were adjusted using the Benjamini-Hochberg correction. Gene sets available from MSigDB were used, from such databases as KEGG, Reactome, BioCarta, WikiPathways, Hallmark, and Gene Onthology. Pathway activity inference of PROGENy was performed with a multivariate linear model (mlm) in decoupler.

Conditional Perturbational Autoencoder was used to learn and explore the perturbation space. We trained the model on study data and the sciPlex 3 dataset, which includes approximately 650,000 cells from three cell lines treated with 188 compounds.

The present example provides the first comprehensive single-cell analysis of PBMC dynamics in patients with IRP undergoing treatment with goflikicept, delineating its cell type-specific effects and revealing its capacity to induce sustained anti-inflammatory reprogramming. While untreated patients with IRP exhibited a PBMC composition broadly similar to that of healthy controls, distinct differences in cell type abundance (e.g., monocytes and other populations) were observed. IL-1 blockade initially elicited a weak anti-inflammatory response and IFN-α response in certain cell types on day 7 of therapy, followed by pronounced anti-inflammatory responses at day 35, particularly in monocytes, characterized by a marked reduction in IL-1β-related signatures. In addition to the prominent effects on monocytes, we observed significant transcriptome changes in B cell subsets (with gene downregulation in naïve B cells and gene upregulation in circulating plasma cell precursors), as well as in MAIT and γδ T cells, suggesting broader effects on adaptive and innate immune responses. Notably, goflikicept clustered pharmacologically with IL-1 inhibitors and tyrosine kinase activity modulators, suggesting a dual targeting of inflammatory pathways. These findings highlight the central role of monocytes in resolving inflammation in IRP, while implicating other immune cell subsets in the therapeutic response.

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

What is claimed is:

1. A method of treating recurrent pericarditis comprising administering subcutaneously to a patient in need of treatment, a loading dose of goflikicept at 240 mg, followed by a maintenance dose of goflikicept at 80 mg once every two weeks.

2. A method of treating recurrent pericarditis comprising administering subcutaneously to a patient in need of treatment, a loading dose of goflikicept at 160 mg, followed by a maintenance dose of goflikicept at 80 mg once every two weeks.
